(19) United States
(12) Patent Application Publication
(10) Pub. No.: US 2013/0292312 A1
(43) Pub. Date: Nov. 7, 2013

(54) BALANCING DEVICE, DIALYSIS MACHINE, EXTRACORPOREAL CIRCULATION AND METHOD FOR BALANCING FLUIDS WITH A FLUID MEASURING CELL

(71) Applicant: Fresenius Medical Care Deutschland GmbH, (US)

(72) Inventors: Alexander HEIDE, Eppstein (DE); Mario KONEGGER, Bad Homburg (DE); Dejan NIKOLIC, Frankfurt (DE); Arne PETERS, Bad Homburg (DE); Christoph WIKTOR, Gelnhausen (DE)

(21) Appl. No.: 13/887,464

(22) Filed: May 6, 2013

Related U.S. Application Data
(60) Provisional application No. 61/642,631, filed on May 4, 2012.

(30) Foreign Application Priority Data
May 4, 2012 (DE) 102012009043.9

(51) Int. Cl. A61M 1/36

(52) U.S. Cl. CPC A61M 1/3663
USPC 210/137; 137/87.03; 137/10

(57) ABSTRACT

A balancing method and a balancing device (100, 101, 200, 201, 301, 303) for determining a fluid balance between a flow quantity in a first flow path (FW1) and a flow quantity in a second flow path (FW2) are disclosed. The disclosed balancing device (100, 101, 200, 201, 301, 303) comprises the following elements:
a differential flow measuring unit (D) for measuring the differential flow between a flow in the first flow path (FW1) and a flow in the second flow path (FW2),
a branch from one of the two flow paths (FW1, FW2) for diverting fluid from one of the two flow paths into the other flow path (W),
a device for setting the flow quantity (P11, P12) in the additional flow path, which can be controlled in such a way that the measured differential flow fulfills a predetermined condition,
   and with a device (K) for determining the flow quantity in the additional flow path as a measure of the fluid balance.

### TECHNICAL FIELD

The invention relates to a balancing device and a method for balancing a fluid in particular a dialysis fluid.

### BACKGROUND

In a method for extracorporeal blood treatment such as hemofiltration, hemodiafiltration, hemodialysis, apheresis and aquapheresis, fluid is normally withdrawn from a patient in a precisely predetermined amount during the treatment. In hemodialysis, blood is circulated in an extracorporeal circulation with a filter, which is divided into two compartments by a semipermeable membrane. The first compartment is connected to the extracorporeal circulation through which the blood flows and the second compartment is connected to a dialysis fluid circulation through which dialysis fluid or dialysate, which is a physiological solution, flows. The amount of dialysis fluid which is carried through the filter in this way is typically 60 to 240 liters per dialysis treatment. The fluid is withdrawn by a pressure gradient through the semipermeable membrane from the blood side to the dialysis fluid side. The quantity of fluid to be withdrawn thus typically amounts to 2 to 5 liters.

It is of crucial importance that the fluid withdrawn is measured with high precision. Withdrawal of even slightly too much fluid could have serious consequences for the patient.

In the machines known previously, either balancing chambers or flow sensors are used for balancing the dialysis fluid. Balancing chambers ensure that the quantity of fluid is identical in two directions, i.e., the quantity of fluids supplied corresponds to the quantity of fluid removed. This is achieved by a chamber having a rigid volume divided into two halves by a flexible gas- and fluid-impermeable membrane, so that each half of the chamber is provided with an inlet valve and an outlet valve that can be cut off. The valves are opened in alternation so that one inlet valve and one outlet valve of the respective other chamber half is opened and closed respectively. The fluid flowing in through the inlet valve causes deformation of the membrane, such that it displaces the fluid into the other half of the chamber and exactly the same amount of fluid flows through the open outlet valve.

A flow path with a delivery device, the so-called ultrafiltration pump, is therefore arranged in parallel with the balancing chamber for the additional withdrawal of fluid from the patient. The fluid to be withdrawn is sent to the balancing chamber past the parallel flow path and is measured by the ultrafiltration pump. Balancing chambers present high demands of the manufacturing tolerance.

Alternatively, flow sensors such as volume or mass flow sensors may be used to detect the inflow quantity and the outflow quantity separately. Thus the quantity of liquid withdrawn is calculated from the difference in the measured flow quantities. The use of volume or mass flow sensors requires a high precision calibration of the sensors at absolute flow rates, such as those described in GB 2003274. This calibration is complex and is usually performed at the plant before delivery of the dialysis machine.

Therefore one object of the invention is to overcome at least one of the aforementioned problems.

### SUMMARY

This object is achieved by a balancing device according to claim 1 and by a method for determining a fluid balance according to claim 17. Advantageous embodiments are defined in the dependent claims.

The differential flow may be expressed as a differential volume or as an integral of a differential flow.

According to one advantageous embodiment, the device for adjusting the flow quantity in the additional flow path includes the device for determining the flow quantity in the additional flow path.

In another embodiment, the device for adjusting the flow quantity in the additional flow path is designed as an adjustable pump.

According to another advantageous embodiment the differential flow measuring unit is embodied as a differential flow sensor for direct measurement of the differential flow between the flow in the first flow path and the flow in the second flow path without a separate measurement of the flow in the first flow path or a separate measurement of the flow in the second flow path.

In another embodiment of the balancing device, it comprises a differential flow sensor having a first flow measuring cell in the first flow path and having a second flow measuring cell in the second flow path through the flow passes in countercurrent with the first flow measuring cell.

In this embodiment, flow measuring cells based on the countercurrent flow principle may be used.

In another embodiment, of the balancing device the additional flow path has a valve that can be cut off. Therefore the additional flow path may be cut off, for example, for calibration purposes.

In another embodiment of the balancing device, the predetermined condition is met when the differential flow is approximately zero.

This is a criterion for regulating the flow in the additional flow path. Furthermore, the flow quantity in the additional flow path indicates directly the differential flow to be measured.

In another embodiment of the balancing device, the device for determining the flow quantity in the additional flow path comprises a container for collecting the flow quantity, and the flow quantity can be determined gravimetrically or by detecting the filling level.

In another embodiment of the balancing device, the additional flow path opens again into one of the two flow paths upstream or downstream from the differential flow sensor and thus forms a parallel flow path to one of the two flow paths.

This yields a closed circuit for the fluid to be balanced.

In another embodiment, the balancing device or a part of the balancing device is designed as a disposable article or as part of a disposable article, advantageously as a flow sensor made of plastic intended for only one use.

In the case of disposable articles, a number of varieties that cannot be controlled at all or completely play a role here such as the storage and shipping conditions and aging. The balancing device that is described can be calibrated for relative flows here. Calibration for absolute flow rates, such as those known in the state of the art would have to take place immediately before use, i.e., the start of treatment of the dialysis treatment in the case of a disposable article for fulfillment of the accuracy requirements. It would be a disadvantage in particular that a precisely known quantity of liquid would have to be passed through the flow sensors. It would be a disadvantage that this flow quantity must be sufficiently large.

Such a flow sensor may in particular be used advantageously for balancing dialysis fluid in mobile or portable dialysis machines or for home dialysis systems. It is advantageous that the manufacturing costs of a flow sensor manufactured in this way may be kept so low that the flow sensor permits disposable use, once per treatment. The flow sensor may be completely or partially integrated into the extracorporeal circulation, for example, in such a manner that the extracorporeal blood circulation has a blood path and a dialysis fluid path such that the dialysis fluid path contains the balancing device described already, used for balancing dialysis fluid in the dialysis fluid path.

For use in dialysis, the embodiment of the balancing device as a disposable article is also advantageous inasmuch as the fact that when the flow sensor is used only once, it does not become covered at all or not significantly with proteins contained in the dialysate. Furthermore, complicated recalibration of the dialysis machine at regular intervals is not necessary.

### BRIEF DESCRIPTION OF THE DRAWINGS

FIG. 1 shows schematically a dialysis machine with a balancing device according to the inventive teaching.

FIGS. 2A and 2B each show a balancing device in according to the inventive teaching in a preferred embodiment.

FIGS. 3A and 3B show additional balancing devices in agreement with the inventive teaching in another advantageous embodiment.

FIGS. 4A and 4B each show a schematic diagram of an arrangement suitable for calibrating a balancing device.

FIG. 5 shows a flow chart of a method for fluid balancing.

FIG. 6 shows another flow chart of a method for fluid balancing.

FIG. 7 shows the principle of the relative calibration on the basis of various flow rates.

### DETAILED DESCRIPTION OF THE DRAWINGS

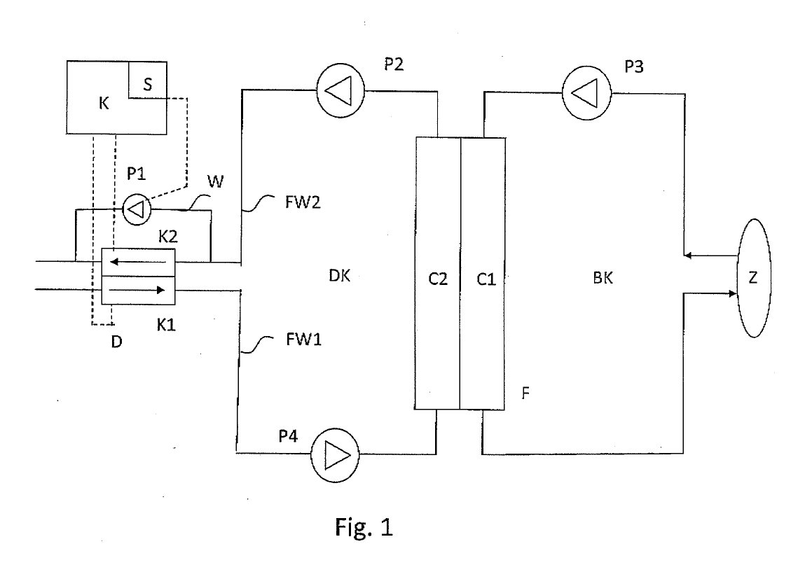

FIG. 1 shows schematically a blood treatment device 1 having a balancing system consistent with the teaching of the present invention. The blood to be treated is withdrawn from the patient via an access Z and is returned by a pump P3 in the blood circulation BK back to the patient through a blood chamber of the filter F and through the access Z. The access Z connects the blood circulation BK to a suitable blood vessel of the patient for taking and returning blood. The access Z may contain a separate outlet and inlet for withdrawing blood and for returning blood (double needle method) or the inlet and outlet may be embodied as a single element (“single needle” method).

A semipermeable membrane in the dialyzer F separates a dialysis fluid chamber C2 from a blood chamber C1. A fluid exchange and mass exchange from the blood chamber C1 into the dialysis fluid chamber C2 take place through the semipermeable membrane. Dialysis fluid is transported through the dialysis fluid chamber C2 of the filter F with a pump P2 downstream from the dialysis fluid chamber and with a pump P4 upstream from the dialysis fluid chamber. The inflow to the dialyzer thus forms a first flow path FW1 and the outflow from the dialyzer thus forms a second flow path FW2. The flow rate in the pump P2 is higher than the flow rate in the pump P4 by the ultrafiltration rate, Due to the difference in flow rates in the pump P2 and in the pump P4, the pressure conditions at the membrane in the dialyzer F are adjusted so that an excess pressure prevails in the blood chamber C1 in comparison with the dialysis fluid chamber C2. Therefore there is a transport of fluid through the membrane from the blood chamber C1 into the dialysis fluid chamber C2, this fluid being known as the ultrafiltrate. The ultrafiltrate amount or ultrafiltration rate can be set by controlling the flow rate of the pump P2 and the flow rate in the pump P4. The flow measuring cells K1 and K2 are connected to a differential flow sensor D, such that the flow measuring cell K1 is situated upstream from the dialysis fluid chamber C2 and the flow measuring cell K2 is situated downstream from the dialysis fluid chamber C2 in the dialysis fluid circulation DK.

The pump P1 forms a flow path parallel to the flow measuring cell K2 in which the fluid transport is controlled by the pump P1.

The differential flow sensor D determines a pair of measured values, consisting of a separate measured value for each flow measuring cell K1, K2, said measured values indicating the flow rate of the fluid through the channel in the respective flow measuring cell. The pair of measured values is preferably determined one or more times per hour and transmitted to a controller K. The controller K assigns a volume flow pair to each measured value pair, wherein mapping of a measured value onto a volume flow may be used, based on a calibration performed previously Alternatively, there may also be mapping onto a mass flow. The controller K derives a control signal for the pump P1 from the volume flow pair thus determined, for example, so that the pump P1 is operated in such a way that the volume flow through both flow metering cells K1 and K2 of the differential flow sensor corresponds at each point in time. For example, the controller K forms the difference from the two volume flows of the volume flow pair and alters the flow rate of the pump P1 by increase or reduction, depending on the sign of the difference in a suitable manner, so that the difference becomes negligible. If the flow through the flow measuring cell K1 is less than the flow through the flow measuring cell K2, this yields a positive value for the difference between the measured values of the flow measuring cell K2 and those of the flow measuring cell K1. The controller K can then modify the control signal for the pump P1 so that the flow rate is increased by the pump P1 and the flow through the flow measuring cell K2 is reduced with an unchanged flow through the pump P2 until the same flow is established as through the flow measuring cell K1. The flow rate through the pump P1 then indicates the differential flow between the flow path emerging from the dialysis fluid chamber and the flow path entering the dialysis fluid chamber. The flow rate through the pump P1 is then a measure of the amount of ultrafiltrate withdrawn in the dialyzer F.

In one embodiment the flow rate through the pump P1 and the flow rate through the pump P4 are each set at a predetermined value, and the flow rate through the pump P2 is regulated by the controller K via a control line to the pump P2 (not shown) so that the differential flow measured in the differential flow sensor D fulfills a predetermined condition such as: becoming negligible.

As an alternative, the flow rate through the pump P1 and the flow rate through the pump P2 may also each be set at a predetermined level and the flow rate through the pump P4 is then regulated by the controller K via a control line (not shown) so that the differential flow measured in the differential flow sensor fulfills a certain condition, for example, becoming negligible.

In these two embodiments, the flow rate through the pump P1 is also a measure of the fluid balance between the first flow path FW1 and the second flow path FW2, i.e., for the amount of ultrafiltrate withdrawn in the dialyzer F.

In another embodiment, the assignment of the measured value pair to a volume flow or a mass flow may be omitted if the difference between the measured values at the same volume flow through both channels is known. In this case the controller K forms the difference from the two measured values and alters the flow rate of the pump P1 by increasing or reducing the difference in a suitable manner until the difference corresponds to the previously known difference at the same volume flow.

The differential flow sensor D may advantageously function according to the magnetic inductive principle in which the two flow measuring cells K1, K2 through which the flow passes in countercurrent have a rectangular cross section and are arranged at a right angle to a magnetic field. The magnetic field is set by the control of the differential flow sensor D and is designed so that a homogeneous field of the same size prevails through both flow measuring cells K1, K2. This is achieved, for example, by the fact that the channels of the flow measuring cells K1, K2 are arranged one above the other in relation to the magnetic field. An electrode is mounted on the inner channel wall, opposite and at a right angle to the magnetic field and to the direction of flow in each channel, extending along the magnetic field. If fluid is flowing through the channel, then a charge separation of the ions present in the fluid is induced by the magnetic field, so that an electrical voltage is applied to the electrodes. This voltage is proportional to the velocity of flow and depends on the magnetic field strength. If the magnetic field is equally great in the two flow measuring cells K1 and K2, then the magnetic field strength dependence for the relative differential flow signal advantageously declines or disappears in forming a differential signal from the two channels.

In other words, disappearance of the differential signal indicates, regardless of the absolute size of the magnetic field in the flow measuring cells K1 and K2, that the flow through the flow measuring cell K1 and the flow through the flow measuring cell K2 are of equal sizes.

The pump P1 is preferably selected from the group of displacement pumps, more preferably a diaphragm pump, a hose roller pump, a piston pump or a geared pump or any other pump which makes it possible to determine the quantity of fluid delivered. For example, the volume delivered with the hose roller pump can be determined with good accuracy from the pump tube volume and the angle of rotation of the rotor of the hose roller pump using known methods. Corresponding methods for determining the quantity of fluid delivered are known from the state of the art for other pumps from the group of displacement pumps.

It is advantageous here that the quantity of fluid to be measured corresponds to the quantity of ultrafiltrate. This quantity is typically 3-5 liters per dialysis treatment or per day, whereas the quantity of dialysate flowing through the flow sensor amounts to a multiple thereof, typically 60-240 liters. Therefore, in agreement with the teaching of the present invention, it is now advantageously possible to use measurement devices or measurement methods for the differential flow, which must have a much lower tolerance than would be necessary for the measurement method, which detects the quantity of dialysate flowing in and flowing out and only then forms a difference.

This is illustrated by the following computation example: for an ultrafiltrate quantity of 5 liters, a measurement error of 5% is equivalent to a quantity of 250 mL as a balance error. If such a measurement method with a measurement error of 5% were used in the dialysate circulation in which the quantity of dialysate flowing in and the quantity flowing out are detected separately, and in which 60 liters of dialysate is delivered through the dialyzer for treatment, then a 5% measurement error would mean a balance error of 3 liters.

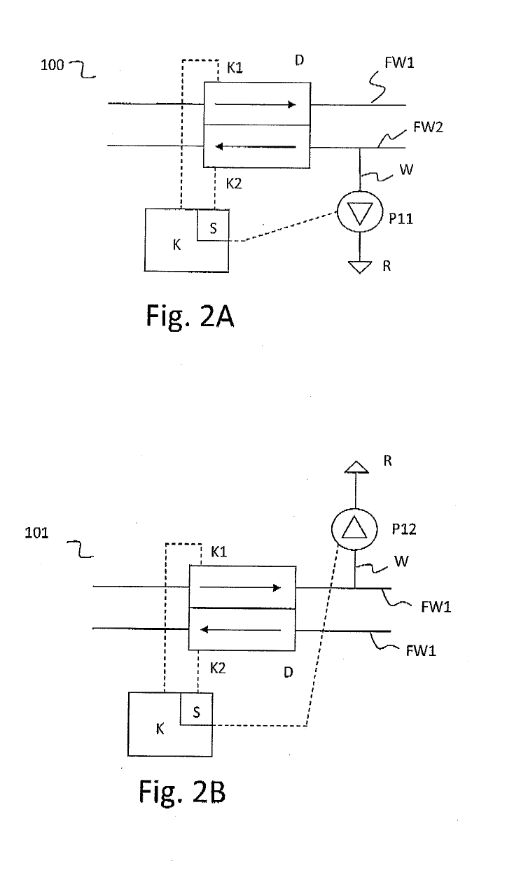

FIGS. 2A and 2B each show a balancing device in accordance with the inventive teaching for determining a fluid balance between a first flow quantity in a first flow path FW1 and a second flow quantity in a second flow path FW2, with a first flow measuring cell K1 in the first flow path FW1 and a second flow measuring cell K2 in the second flow path FW2, where one of the two flow paths FW1, FW2 comprises a branch for diverting fluid into another flow path W. The same reference numerals as those used and introduced in conjunction with FIG. 1 indicate the same or corresponding elements in FIGS. 2A and 2B. In the balancing devices of FIG. 2A, the additional flow path W branches off from the second flow path FW2, and in FIG. 2B the additional flow path W branches off from the first flow path FW1. The balancing devices in FIGS. 2A and 2B each have a device for adjusting the flow quantity in the additional flow path W, namely each having a pump P11 and/or P12. The device for adjusting the flow quantity in the additional flow path can be controlled in such a way that the measured flow quantity of the first flow measuring cell K1 and the second flow measuring cell K2 fulfills a predetermined condition, preferably that the differential flow between a flow in the first flow path FW1 and a flow in the second flow path FW2 fulfills a predetermined condition such as that the differential flow is zero or approximately zero. The device for determining the flow quantity P11, P12 in the additional flow path—in other words, the adjustable pump P11 or the adjustable pump P12—serves as a measurement unit for the fluid balance.

In the application as a fluid balancing system for dialysis, the first flow path FW1 is an inflow to a dialysis fluid chamber of a dialyzer, the second flow path FW2 is the outflow from the dialysis fluid chamber and the fluid balance is a measure of the quantity of ultrafiltrate withdrawn.

The two flow measuring cells may be combined to a differential flow sensor D, for example, the different flow sensor described in GB 2003274. With the differential flow sensor described in GB 2003274, the flow measuring cells operate according to the magnetic inductive principle, in which both flow measuring cells are exposed to the same magnetic field so that variations in the magnetic field strength act equally on the two flow measuring cells. The fluid flowing through the flow measuring cell at a right angle to the magnetic field experiences a charge separating effect In accordance with the Lorentz force so that a voltage can be measured on the electrical contacts of the flow measuring cell arranged essentially at a right angle to the magnetic field and to the direction of flow. The fluid must contain electrically charged ions or dissociated molecules, which is typically the case in the dialysis fluid. This requirement is not necessary for other flow measuring cells which do not operate according to the magnetic inductive measuring method.

This device, which is suitable for adjusting the flow quantity in the additional flow path, may be designed as a pump P11, P12, as shown in FIGS. 2A and 2B.

A container, preferably a bag, may be connected to the outlet R, this container being attached so that it is suspended or hanging freely on a balance, and it collects the quantity of fluid conveyed through the additional flow path. Such a bag may at the same time also serve as a device for determining the flow rate in the additional flow path as well as a device for collecting the fluid quantity, wherein the fluid quantity is determined gravimetrically using the scales. It is thus possible to detect the fluid quantity in this way. But this arrangement in comparison with existing systems for fluid balancing with separate bags for the inflow and outflow, it is advantageous with this arrangement that the bag described can be very small and can be mounted accordingly in a location on the device that is protected from mechanical effects. This also advantageously prevents interference with the scales for the bag and thus also the balancing when changing the dialysis fluid bag during a treatment. Balancing with scales has previously preferably been used in acute dialysis therapy.

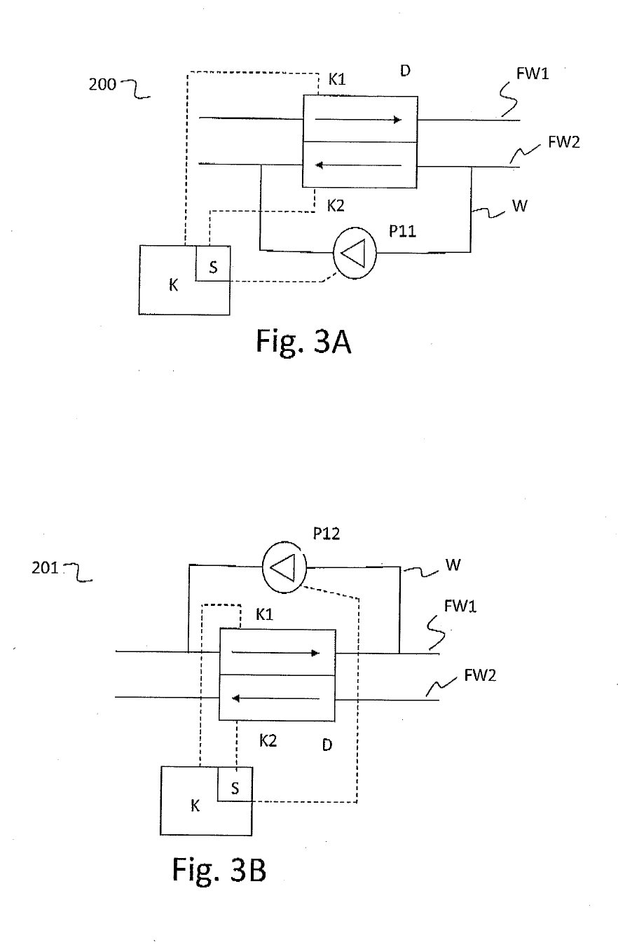

FIGS. 3A and 3B show additional balancing systems in three preferred types of embodiments in accordance with the teaching of the present invention. The same reference numerals as in FIGS. 1 and 2A and 2B indicate the same or corresponding elements.

The balancing systems shown in FIGS. 3A and 3B have in common the fact that the additional flow path W upstream from the first flow measuring cell K1 (in the exemplary embodiment in FIG. 3B) and/or downstream from the second flow measuring cell K2 (in the exemplary embodiment of FIG. 3A) again opens into the flow path of the respective flow measuring cell and thus forms a parallel flow path to this flow measuring cell. The balancing systems shown in FIGS. 3A and 3B each have a differential flow sensor D with first and second flow measuring cells K1 and K2 through which the flow passes in countercurrent. A fluid, namely a dialysate in a preferred embodiment, flows through the first flow measuring cell K1 at a first flow rate. In the embodiment in FIG. 3A the additional flow path W branches off upstream from the second flow measuring cell K2, passes through the pump P11 and thus branches off downstream from the flow measuring cell K2, thereby forming a flow path parallel to the flow measuring cell K2. In the embodiment shown in FIG. 3B the additional flow path W branches off downstream from the first flow measuring cell K1, passes through pump P2 and again opens into the first flow path FW1 upstream from the first flow measuring cell K1 thereby forming a flow path parallel to the first flow measuring cell K1. In this way the fluid is passed by the second flow measuring cell K2 under the control of pump P11 and/or is returned under the control of the pump P12.

In the application as a flow balancing system for dialysis, additional components (not shown) may be present in the fluid path FW1 and/or in the fluid path FW2, for example, an air separation chamber or a heater for the dialysate in the fluid path FW1, between the differential flow sensor and the pump P11 and/or between the differential flow sensor and the pump P12, which functions here as a ultrafiltration pump.

The controller K comprises a data memory S and is connected to the pump P11 and/or the pump P12 so that the controller K can adjust the flow rate of the pump P11 and/or of the pump P12. The connection may also be suitable for determining or regulating the flow rate.

In addition the controller K is connected to the differential flow sensor ID via one or more lines. The differential flow sensor ID may perform a preprocessing of the measurement signal. In particular the differential flow sensor may transmit one measured value per flow measuring cell K1, K2 separately or as a measured value pair or may transmit one measured value of the differential flow to the controller K accordingly. The differential flow sensor D determines prevailing measured values preferably at discrete intervals, more preferably once per second, even more preferably several times per second. The controller determines a control signal for the pump P11 and/or for the pump P12 based on the measured value obtained by the differential flow sensor D and thus forms a control circuit.

In an exemplary embodiment the differential flow sensor D transmits one measured value per flow measuring cell K1, K2, i.e., one measured value pair to the controller K at a certain point in time. The controller K determines a control signal for adapting the flow rate of the pump P11 and/or the flow rate of the pump P12 from the parameters known from calibration or with the help of mapping.

In the exemplary embodiment depicted in FIG. 3A, fluid is conveyed through the flow path parallel to the flow measuring cell K2 in the same direction of flow as in the flow measuring cell K2 through the pump P11. This arrangement may be used in particular when the flow through the second flow path FW2 is greater than the flow through the first flow path FW1, for example, when the flow measuring cell K2 is arranged downstream from a dialyzer and the ultrafiltrate is added to the flow through the first flow path FW1.

In the exemplary embodiment shown in FIG. 3B, fluid is conveyed through the additional flow path FW parallel to the flow measuring cell K1 opposite the direction of flow in the flow measuring cell K1 through the pump P12. This arrangement may be used in particular when the flow through the second flow path FW2 is less than the flow through the first flow path FW1. This is the case, for example, when the flow measuring cell K1 is arranged upstream from the dialyzer and the flow through the second flow path is greater due to the ultrafiltrate.

In another advantageous embodiment, a return valve may be arranged in the flow path of the flow measuring cell K2 to allow fluid transport to occur only in the direction intended.

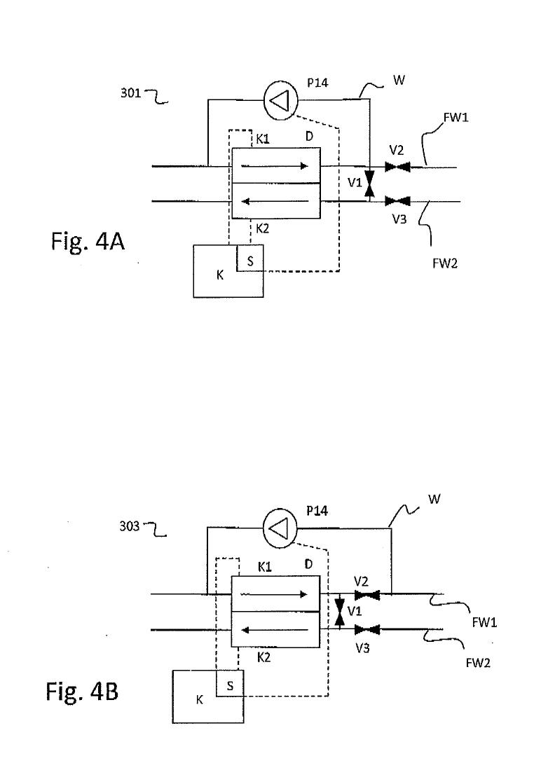

FIGS. 4A and 4B each show schematically an arrangement for performing the calibration. The arrangements shown in FIGS. 4A and 4B each supplement the arrangement of FIG. 3B by the addition of valves V1, V2 and V3.

The calibration may advantageously be performed immediately before the treatment. In addition the calibration may advantageously be performed without a previously known flow quantity. For the calibration, the valves V2 and V3 are closed and the valve V1 is opened. The pump P14 is put in a state so that no fluid can flow through the pump 14.

Depending on the pump 114 used, an additional closable valve not shown in FIGS. 4A and 4B upstream or downstream from the pump P14 may also be closed.

In an advantageous embodiment of FIG. 4B, the valve V3 is arranged so that by opening valve V1 and at the same time closing the two valves V2 and V3, a flow path is formed without a branch from flow measuring cell K1 to flow measuring cell K2. Pump P14 may also be used advantageously and according to the embodiment in FIG. 4B for the fluid transport through the two flow measuring cells K1 and K2 during calibration.

It is important and is ensured by the arrangements shown in FIGS. 4A and 4B that the two channels of the differential flow sensor D form a continuous flow path so that the same flow quantity flows through the two flow measuring cells K1 and K2.

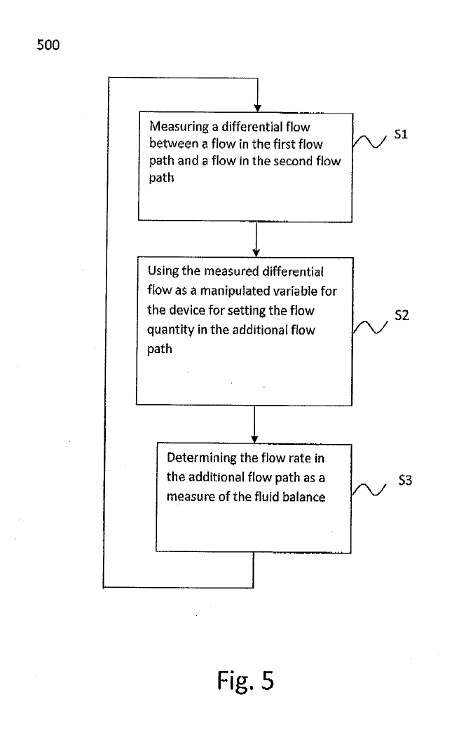

FIG. 5 shows a method for determining a fluid balance between a flow quantity in a first flow path and a flow quantity in a second flow path in accordance with the teaching of the present invention. The method according to the invention may advantageously be performed with one of the balancing devices described in conjunction with FIGS. 1, 2A, 2B and 3B.

This method comprises the following steps:

S1: Measuring a differential flow between a flow in the first flow path and a flow in the second flow path
S2: Using the measured differential flow as a manipulated variable for fulfilling a predetermined condition for the equipment for setting the flow quantity in the additional flow path and
S3: Setting the flow rate in the additional flow path using the device, determining the flow rate and using the flow rate to derive a measure for the fluid balance.

The flow measuring cell may be a differential flow sensor D as shown in FIG. 1, FIG. 2A, 2B, 3A, 3B, 4A or 4B, but other volume or mass flow sensors may also be used wherein the differential flow signal is obtained only in post-processing of the individual flow signals. The flow measuring cells may already detect the measured value and preprocess it and transmit the value thereby obtained to the controller K.

The controller K with the memory S receives the two measured values and assigns flow quantities to the parameters in the memory known from the calibration. The assigned flow quantities need not necessarily correspond to the actual absolute flow quantities but they must be correct only in comparison with one another, i.e., in relation to one another. The correspondence may take place via a linear mapping or another suitable form of mapping, wherein the parameters in the memory S are then the parameters in the map. However, the parameters in the memory S may also be assigned to functions or groups by sections, so that the calibration is composed piece by piece of different maps in certain flow rate ranges over the entire flow rate range.

The values obtained in this way are linked in the controller K and a manipulated variable for the device for setting the flow quantity in the additional flow path is determined with this predetermined condition. This linking is advantageously embodied as the formation of a difference or a sum. The manipulated variable is output by the controller in the form of a signal. The device for setting the flow rate in the additional flow path receives the signal and alters the flow rate accordingly. The signal may be a digital or analog signal. The device for setting the flow rate may be designed here as an adjustable pump.

The predetermined condition is met, for example, when the flow quantity through the first and second flow paths is the same. The method and device according to the invention the same under any other predetermined conditions when the calibration for both flow quantities has been performed and the flow quantity can be kept constant in one of the two flow paths without the additional device for setting the flow quantity and the additional flow path.

Steps S1, S2 and S3 of the method described here are repeated in this order. Steps S1, S2 and S3 are preferably performed at least once per second, more preferably being repeated several times per second.

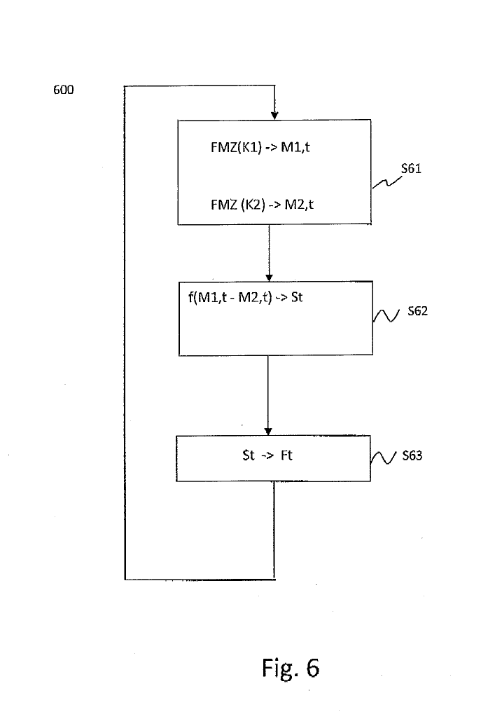

FIG. 6 shows the method according to the invention in another advantageous embodiment. This method functions like the method described in conjunction with FIG. 5, whereby the measured values M1t and M2t are assigned to first and second flow measuring cells FMZ1 and FMZ2 in a first step S61, said cells determining the flow quantity in a first and second flow paths K1 and K2, respectively, and the respective measured values M1t, M2t are assigned to a corresponding point in time t. In step S62, the controller K with the memory S forms the difference in the two measured values and with the help of the known parameters from the calibration in the memory S, it forms a manipulated variable St for the device for setting the flow quantity in the additional flow path. In step S63, the controller K outputs the manipulated variable in the form of an analog or digital signal to the device for setting the flow quantity Ft in the additional flow path. These method steps are repeated periodically in time, advantageously once per second, preferably several times per second.

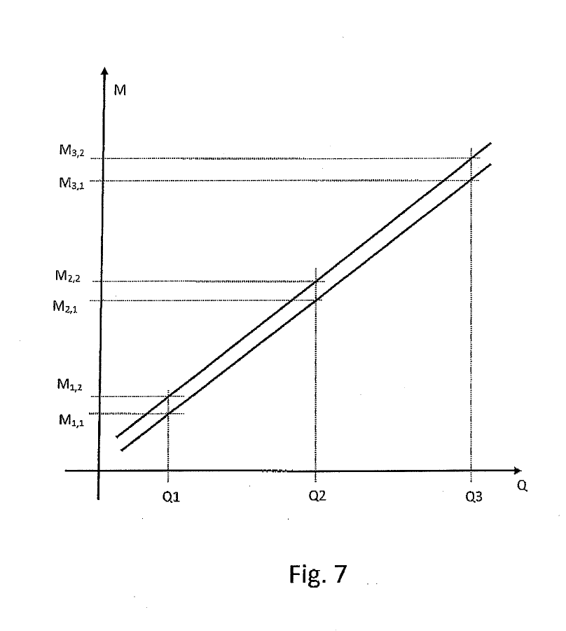

A method for calibrating a balancing device as shown in FIGS. 4A and 4B is illustrated in FIG. 7.

Fluid is pumped by means of a pump, not shown in FIGS. 4A and 4B, through the two flow measuring cells K1 and K2 at the same predetermined flow rate, which is not necessarily known. The measured value determined by the differential flow sensor D per flow measuring cell (K1, K2) is transmitted to the controller K. This relationship is shown in FIG. 7 as an example, where the predetermined flow rate Q1 which is not known more precisely is set and advantageously corresponds to the flow rate for the dialysate during the treatment. The differential flow sensor D determines a measured value M1,1 for the first flow measuring cell K1 and the measured value M1,2 for the second flow measuring cell K2 and transmits the measured value pair to the controller K. The measured values thereby determined need not indicate the actual absolute flow rate through the respective flow measuring cell.

The controller K stores both values in the memory unit S. In another embodiment, the controller forms the difference from the two values, for example, and stores the differential value in the memory unit S. This invention is not limited to these two exemplary embodiments and also includes additional embodiments.

In an advantageous refinement, the calibration is repeated with several different flow rates Q1, Q2 and Q3 and the values or value pairs thereby determined (M2,1; M2,2 and M3,1; M3,2) are stored separately in the memory S. The pumps P2 and P4 in FIG. 1 are advantageously used for the dialysate circuit DK in FIG. 1, typically peristaltic pumps, and their flow rate is determined with a method known from the state of the art and reported to the controller K. It is important here to select the various flow rates to detect the entire range used during the treatment, for example, 100 mL/min, 200 mL/min, 500 mL/min.

It has been found that the relationship between the measured value and flow rate is essentially linear. The controller K advantageously calculates the respective measured value pair for a dialysate flow rate set during the treatment by using a linear interpolation.

For proper functioning of the balancing system according to the invention, it is not absolutely essential for the two channels to have the flow passing through them in opposite directions.

The method described here and the balancing system described here also function with other flow sensors, for example, electric inductive sensors, Coriolis sensors and flywheel sensors as well as other flow sensors which are known from the state of the art. Mass flow sensors are especially advantageously used to minimize or rule out measurement errors due to air bubbles in the fluid. The flow sensor may perform a preprocessing of the measurement signals by the measurement units, for example, electrodes for electrically inductive flow sensors, and to transmit a digital or ratiometric output signal to the controller K.

The method and the balancing systems described here corresponding to one of the embodiments from FIGS. 2A, 2B, 3A, 3B, 4A and 4B may also be designed so that the flows through the flow measuring cell K1 and the flow measuring cell K2 are offset in time from one another and the differential flow is expressed as a differential volume, as an integral of a differential flow or as a difference of an integral of the flow in the first flow path and an integral of the flow in the second flow path. For example, liquid is transported first through flow measuring cell K1 and flow measuring cell K2 does not transport any liquid. The controller K records as an example the measured values or measured value pairs, which are transmitted by the flow sensor during this period of time. If, in a second period of time, the fluid transport through the flow measuring cell K2 is stopped and fluid is transported through flow measuring cell K1, then the controller can replace the measured value of the flow measuring cell K1 by the value recorded previously and can control the flow rate of the pump P1 with the newly formed measured value pair. The method of balancing with the balancing system described here may also be designed so that the respective flow measuring cell transports fluid at a predetermined flow rate instead of not transporting any fluid at all and it is assured that this predetermined flow rate is the same in both periods of time. This offset in flows through flow measuring cell K1 and flow measuring cell K2 may be used to particular advantage in peritoneal dialysis.

1. A balancing device (100, 101, 200, 201, 301, 303) for determining a fluid balance between a flow quantity in a first flow path (FW1) and a flow quantity in a second flow path (FW2), comprising
a differential flow measuring unit (D) for measuring the differential flow between a flow in the first flow path (FW1) and a flow in the second flow path (FW2),
a branch from one of the two flow paths (FW1, FW2) for diverting fluid from one of the two flow paths into the other flow path (W),
a device for setting the flow quantity (P1, P2, P4, P11, P12) in one of the two flow paths and/or in the additional flow path, which can be controlled in such a way that the measured differential flow fulfills a predetermined condition,
and with a device (K) for determining the flow quantity in the additional flow path as a measure of the fluid balance.

2. The balancing device (100, 101, 200, 201, 301, 303) according to claim 1, wherein the device for setting the flow quantity (P11, P12) in the additional flow path (W) includes the device for determining the flow quantity in the additional flow path (P11, P12).

3. The balancing device (100, 101, 200, 201, 301, 303) according to claim 1, wherein the device for setting the flow quantity (P1, P2, P4, P11, P12) in one of the two flow paths and/or in the other flow path is designed as an adjustable pump (P11, P12).

4. The balancing device (100, 101, 200, 201, 301, 303) according to claim 1, wherein the differential flow measuring unit (D) is designed as a differential flow sensor for direct measurement of the differential flow between the flow in the first flow path and the flow in the second flow path without a separate measurement of the flow in the first flow path or a separate measurement of the flow in the second flow path.

5. The balancing device (100, 101, 200, 201, 301, 303) according to claim 4, wherein the differential flow sensor has a first flow measuring cell (K1) in the first flow path (FW1) and a second flow measuring cell (K2) in the second flow path (FW2), in the second flow path (FW2), the flow passing through the flow measuring cell in countercurrent with the first flow measuring cell.

6. The balancing device (100, 101, 200, 201, 301, 303) according to claim 1, wherein the differential flow measuring unit comprises a first flow sensor K1 for measuring the flow in the first flow path and a second flow sensor K2 for measuring the flow in the second flow path.

7. The balancing device (100, 101, 200, 201, 301, 303) according to claim 6, wherein the first and/or the second flow sensor is a flow measuring cell designed as a volume flow sensor or a mass flow sensor.

8. The balancing device (100, 101, 200, 201, 301, 303) according to claim 1, wherein the additional flow path has a cutoff valve.

9. The balancing device (100, 101, 200, 201, 301, 303) according to claim 1, wherein the predetermined condition is satisfied when the differential flow is approximately zero.

10. The balancing device (100, 101) according to claim 1, wherein the device for determining the flow quantity in the additional flow path comprises a container for collecting the flow quantity, and the flow quantity can be determined gravimetrically or by filling level detection.

11. The balancing device according to claim 10, wherein the container is a bag.

12. The balancing device (200, 201, 301) according to claim 1, wherein the additional flow path W upstream or downstream from the differential flow sensor D opens back into one of the two flow paths upstream or downstream and thereby forms a parallel flow path to one of the two flow paths.

13. The balancing device according to claim 1, wherein the differential flow is determined as a flow rate or as a flow volume.

14. The balancing device according to claim 1, partially or completely embodied as a disposable article or as part of a disposable article.

15. The balancing device according to claim 1, wherein the differential flow can be measured as a differential volume, as an integral of a differential flow or as the difference between an integral of the flow in the first flow path and an integral of the flow in the second flow path.

16. A dialysis machine comprising a balancing device according to claim 1 for balancing dialysis fluid.

17. An extracorporeal blood treatment unit having a blood path and a dialysis fluid path containing a balancing device according to claim 1 for balancing dialysis fluid in the dialysis fluid path.

18. A method (500, 600) for determining a fluid balance between a flow quantity in a first flow path and a flow quantity in a second flow path, having a balancing device according to claim 1, wherein the method comprises the following steps:
measuring a differential flow between a flow in the first flow path and a flow in the second flow path (S1, S62),
using the measured differential flow as a manipulated variable for the device for setting the flow quantity in the additional flow path (S2, S63) and
determining the flow rate in the additional flow path as a measure of the fluid balance (S3).

19. The method according to claim 18, wherein the predetermined condition is met when the differential flow is approximately zero.
